(19) United States
(12) Patent Application Publication
(10) Pub. No.: US 2012/0263668 A1
(43) Pub. Date: Oct. 18, 2012

(54) GEL COMPOSITION

(75) Inventors: Alison Margaret Cowan, Bramhall (GB); Meiki Maggie Wu, Manchester (GB); Paul Lawrence Heffernan, Northwich (GB); Helen Patricia Atkinson, Gatley (GB)

(73) Assignee: PZ CUSSONS (UK) LIMITED, Manchester (GB)

(21) Appl. No.: 13/500,285

(22) PCT Filed: Oct. 11, 2010
(86) PCT No.: PCT/GB2010/051706

§ 371 Date: Jun. 25, 2012

(30) Foreign Application Priority Data
Oct. 9, 2009 (GB) 0917736.1

(51) Int. Cl. A61K 8/73; A61Q 9/02; A61P 19/00; A61K 8/42

(52) U.S. Cl. 424/70.13; 514/788; 510/135; 510/119

(57) ABSTRACT

The present invention relates to a stable, post-foaming gel composition for use in personal cleansing which is able to enhance the sensory experience for a consumer by remaining adhered to the consumer's skin for longer before being washed off, enabling greater levels of, for example, skin moisturisation and fragrance, to be delivered to the skin, and is also able to exhibit enhanced stability and clarity.

The present invention relates to a stable, post-foaming gel composition for use in personal cleansing which is able to enhance the sensory experience for a consumer by remaining adhered to the consumer's skin for longer before being washed off, enabling greater levels of, for example, skin moisturisation and fragrance, to be delivered to the skin, and is also able to exhibit enhanced stability and clarity.

Applying non-post foaming liquid products under a constant bombardment of water in, for instance, a shower, can be frustrating for consumers because most of the product is washed away before a satisfactory washing experience is achieved. Much of the pleasure of washing with soap bars comes from the engagement with the bar as a creamy lather is generated. Whilst a creamy lather is easier to obtain using soap bars, delivery of moisturisers or fragrance to the skin is restrictive. It is difficult to deliver improved skin moisturisation and fragrance delivery from bar formats as often the materials included for moisturisation and fragrance are bound within the bar structure format and are not available for efficient delivery to the skin.

Post-foaming gel compositions are known in the art. For example, U.S. Pat. No. 4,772,427 to Dawson describes a post-foaming gel composition which is able to incorporate higher levels of foaming agent within the gel structure, the intention being to optimise foaming characteristics. This composition comprises a major amount of water, about 3-23% by weight of a water-soluble anionic alkali metal C₁₀-C₁₆ alkyl ether sulfate surfactant, about 1-24% by weight of a water dispersible ethoxylated fatty alcohol or fatty ester, about 2-4% of isopropyl myristate, about 1-10% of a mono- or disaccharide and about 5-20% by weight of a saturated aliphatic hydrocarbon foaming agent having 4 to 5 carbon atoms as an integral part of the gel structure, the anionic surfactant and the ethoxylated fatty alcohol or ester being present in a weight ratio of about 4:1 to about 1:4.

EP 1295588 to Mibelle AG Cosmetics describes a post-foaming gel composition which uses thickening agents in the composition, the thickening agents used being intended to prevent the compositions from being too thick and viscous, as they are said to be previously. The composition comprises 0.5-9% by weight of at least one alkoxylated amide as a non-ionic surface active thickener and at most 0.1% by weight of polymeric thickeners. A key benefit of using post foaming shower gels is the consumer satisfaction achieved as through generation of a thick, rich creamy lather. However, many people still prefer to use a conventional bar of soap rather than a shower gel. It is believed that one factor responsible for resistance to the use of shower gels is connected to lather generation. In order to produce lather from a shower gel the user must apply shear to the gel; however, this effort is wasted in many cases. This is because the lather washes away before it can be applied to any significant part of the body. In contrast, a steady supply of lather can be obtained from using a bar of soap.

Standard shower gels can be either too thin or too thick in their consistency, and have an inherent disadvantage because of their poor adherence to skin, giving rise to short skin contact times and hence poor skin moisturisation and fragrance delivery, poor foaming characteristics and/or more product needing to be used for each wash.

Post-foaming shower gels have been developed to address these problems. However, it has been found that a particular attribute, herein defined as gel rigidity, is a critical factor affecting product performance. If the gel is too rigid on dispensing it falls or is washed off the skin before foam can be generated effectively. On the other hand, if the product is too thin, it can run off the skin before the user can generate a lather. Additionally the foaming agent is not sufficiently entrapped within the gel and leaks out, causing a poor foaming performance. It has further been the case that some existing post-foaming shower gels suffer from a lack of stability. This is realised in the diffusion of a thin liquid from the gel upon being dispensed from its container. It is believed that this is at least in part attributable to poor levels of entrapment of the post-foaming agent in the gel. Clearly, unstable gels of this nature will not be able to produce foam or lather as efficiently as a stable gel if the post-foaming agent is leaking from the product.

It is believed that the clarity of a gel is indicative of how stable it is; the clearer the gel, the more stable it is. The gels which are more hazy or cloudy in their appearance are indicative of the formulation not adequately holding the post-foaming agent within the gel. Additionally, in general, from an aesthetic viewpoint, consumers prefer gels which are clear in their appearance over gels which are more opaque.

None of the shower gel compositions currently available possess a satisfactory combination of gel rigidity for an optimum skin contact time for lather generation and transfer of fragrances and moisturisers, stability against separation of the post-foaming agent from the gel, and gel clarity.

It would therefore be desirable to provide a post-foaming gel composition which is able to overcome one or more of the problems detailed above.

Therefore, according to the present invention there is provided a post-foaming cleansing composition comprising:

i) an anionic surfactant;

ii) one or more non-ionic surfactants;

iii) a polymeric thickener; and

iv) a post-foaming agent

wherein the polymeric thickener is present in an amount of at least 0.3% by weight of the composition, wherein the anionic surfactant is present in the composition relative to the one or more non-ionic surfactants in a ratio of no more than 4.0:1, and wherein the one or more non-ionic surfactants include a fatty acid alkanolamide.

According to one embodiment of the invention, the fatty acid alkanolamide is a polyethylene glycol fatty acid amine, for example a polyethylene glycol-4 fatty acid amine. A typical fatty acid alkanolamide used in accordance with the invention is polyethylene glycol-4 rapeseedamide (PEG-4 rapeseedamide), which is sold under the trade name Amidet N.

The composition of the invention as defined hereinabove is able to provide optimum clarity as well as optimum rigidity for maximum adherence to a person's skin, allowing for a prolonged contact time with the skin.

It has been discovered that there is an is an optimum level of gel rigidity for post foaming gels and an optimum gel structure for the delivery of a post foaming gel which is capable of releasing and delivering fragrance and skin conditioning agents (e.g. moisturisers) more efficiently than either from a bar or standard shower gel because of its improved contact time with the skin.

If a gel composition has the determined optimum rigidity, it is able to adhere to the skin for a longer period of time without running, falling or being washed off, leading to an improved sensory in-use experience whilst showering. A further advantage of the composition of the invention derived due to the improved adherence of the composition to the skin is that consumers are able to achieve a much more efficient utilisation of their post-foaming shower composition because the amount of composition required to achieve a desired amount of foam for each wash is much less than for a standard shower gel. A still further benefit is that less of the gel composition is needed to have a satisfactory wash as the product is effectively more concentrated, leading to a reduced environmental impact with fewer chemicals being released into the environment. The composition is also better value than existing standard shower gels as more washes can be obtained from one container of the composition.

By “polymeric thickener”, it is intended to mean any substance having a polymeric structure which is able to increase the viscosity of a composition when added thereto. The polymeric thickener contributes in particular to the creaminess and longevity of the foam which is produced during washing to provide an optimum lather texture during the washing. It also contributes to the increased skin adhesion of the compositions.

The minimum amount of polymeric thickener which is to be used in the composition of the invention is about 0.3% by weight of the composition. This is in stark contrast to the existing art such as EP 1295588 which strongly teaches against using any more than 0.1% by weight of polymeric thickener in a post-foaming composition, while the Dawson reference does not advocate the use of a polymeric thickener at all.

Typically, the composition contains at least about 0.4% by weight polymeric thickener, more typically at least about 0.5% by weight.

According to one aspect of the invention, the composition contains up to about 3.0% by weight of polymeric thickener therein, depending upon the molecular weight and nature of the thickener. Typically, however, up to about 1.0% by weight of polymeric thickener is used.

The polymeric thickeners which are used in the composition of the invention should ideally not compromise the clarity of the gel compositions.

Typical polymeric thickeners which may be used in the composition of the invention include, but are not limited to, cellulose based thickeners such as hydroxypropylmethyl cellulose (such as e.g. Walocel® series ex-Dow Wolff Cellulosics or Benecel MP333C ex-Aqualon), sodium carboxymethyl cellulose (e.g. Cekol 30,000G ex-CP Kelco), hydroxyethyl cellulose (e.g. Natrosol HHR ex-Aqualon), hydrophobically modified HEC (e.g. Natrosol Plus 330 ex-Aqualon), hydroxypropyl cellulose (e.g. Primaflo MP 3295A ex-Aqualon). A further class of polymeric thickeners includes natural gums and their derivatives (via chemical modification) such as xanthan gum (e.g. Keltrol CORD), carrageenan gum (e.g. Genuvisco CI 123), gellan gum (e.g. Kelcogel CGHA), all ex-CP Kelco, and hydroxypropyl guar (e.g. N-Hance HP 40S ex-Aqualon) and cationic guars (e.g. N-Hance 3000 series ex-Aqualon). A further class of polymeric thickeners are the synthetic type such as carbomers (e.g. Carbopol ETD 2020 ex-Lubrizol), acrylate copolymers (e.g. Synthalen W400 ex-3V), hydrophobically modified alkali soluble emulsion polymers (e.g. Aculyn 22 ex-Rohm & Haas), hydrophobically modified ethoxylated urethanes (e.g. Aculyn 44 ex-Rohm & Haas) and polyethylene glycol (PEG) based nonionic polymers such as PEG-150 distearate and PEG-120 methyl glucose dioleate (e.g. Glucamate DOE ex-Lubrizol).

While a polymeric thickener such as a starch could be used in the composition of the invention, it can compromise the clarity of the gel compositions. Therefore, according to one embodiment of the invention, no starch is used in the compositions.

According to a further aspect of the invention, the ratio of anionic surfactant to non-ionic surfactant in the composition is typically less than 4.0:1, more typically between about 3.2:1 and about 3.9:1, still more typically between about 3.3:1 and about 3.8:1, still more typically between about 3.4:1 and about 3.8:1, still more typically between about 3.6:1 and about 3.7:1. Most typically, the ratio is about 3.7:1.

Typical anionic surfactants which may be used in the composition of the invention include but are not limited to, alkali metal alkyl sulphates (e.g. sodium coca sulfate), alkali metal alkyl ether sulphates (e.g. sodium lauryl ether sulphate (SLES)), sulfosuccinates, acyl glutamates, sultaines, taurates, carboxylates, isethionates, alkyl phosphates, sarcosinates (e.g. Crodasinic LS35 ex-Croda), olefin sulphonates and alkyl polyglucoside esters (e.g. Eucarol AGE range ex-Lamberti).

Typical non-ionic surfactants which may be used in the composition of the invention in addition to the fatty acid alkanolamide include, but are not limited to, one or more selected from alkoxylated alcohols, glyceryl esters (e.g. glyceryl oleate or PEG-7 glyceryl cocoate), glycol esters, alkyl poly glucosides, alkoxylated carboxylic acids, other alkanolamides and their derivatives. Typical non-ionic surfactants include alkoxylated alcohols such as laureth-2, laureth-4, C12/13 pareth-3, ceteareth-4 or oleth-3 or glycol esters such as coconut fatty acid monoglyceride polyglycol ether or modified palm oil polyglycol ether.

According to one embodiment of the invention, the one or more non-ionic surfactants comprise a combination of laureth-4 and PEG-4 rapeseedamide.

The composition of the invention also contains a post-foaming agent to provide the foaming effect. The foaming agent is typically a saturated aliphatic hydrocarbon compound having 4 or 5 carbon atoms. Typical examples of post-foaming agents include, but are not limited to, one or more compounds selected from n-pentane, isobutane, n-pentane and isopentane. Isopentane is typically used.

Typical amounts of the post-foaming agent in the composition of the invention are from about 3 to about 15 wt. % of the overall composition, more typically from about 8 to about 13 wt. % of the overall composition.

It has been established by the inventors that for a most advantageous level of gel rigidity, clarity and foaming agent entrapment (i.e. composition stability) in a typical composition according to the invention, the anionic surfactant is SLES, the non-ionic surfactant is a combination of laureth-4 and PEG-4 rapeseedamide, the polymeric thickener is a hydroxypropylmethyl cellulose-based thickener, the amount of polymeric thickener is about 0.5% by weight of the composition, and the ratio of anionic surfactant to non-ionic surfactant is about 3.7:1.

The laureth-4 is important for the stability of the entrapment of the post-foaming agent in the gel, while the PEG-4 rapeseedamide is important for providing clarity in the gel formulation.

In order to achieve the desired levels of rigidity, stability and clarity simultaneously, it has been determined that appropriate relative amounts of the anionic and non-ionic surfactants are required, i.e. a ratio of no more than 4:1, together with the appropriate quantity of polymeric thickener.

Compositions, such as those detailed in WO97/03646, may comprise an anionic surfactant, a non-ionic surfactant and a polymeric thickener, but the need for the correct balance between the anionic and non-ionic surfactants was not recognised, as is apparent from the compositions having an anionic:non-ionic surfactant ratio of 17.9:1.

According to a further aspect of the invention, the composition may also contain one or more amphoteric surfactants which may include, but are not limited to, capryl/capramidopropyl betaine (such as Tegobetaine 810), laurylamidopropyl betaine, cocoamidopropylbetaine, alkyl betaines, alkyl amine oxides, alkyl amidopropyl amine oxides, amphoacetates/diacetates, amphopropionates and alkyl amidopropyl hydroxysultaines.

According to a further aspect of the invention, the composition may also contain one or more additional components which are conventionally used in cleansing compositions and which are able to impart one or more desired characteristics upon the composition, and which do not adversely affect the structure. Exemplary suitable additional components include, but are not limited to, fragrances, skin conditioning agents, colouring agents, preservatives, antiseptic agents, chelating agents, sunscreen agents, plant extracts, pearlisers, opacifiers, shimmering agents, exfolliants, silicone oils, lipids, vitamins, skin lightening agents, pH adjusters, and the like. These additional components typically constitute between about 2 and about 5% by weight of the composition.

The balance of the composition is typically made up to 100% weight by deionised water.

The pH of the composition is typically in the range of from about 4.0 to about 9.5, depending upon the precise formulation.

The optimum desirable level of gel rigidity is from about 3 to about 3.5 for optimum adherence to the skin for lathering and foam generation, although levels from about 2.5 to about 4 can also produce satisfactory results. The gel rigidity level is assessed using the scale detailed below.

Rating of gel rigidity:

1=runny, insufficient gel structure, runs off skin easily

2=very soft gel, not self-supporting under gravity

3=soft but self-supporting gel with peaking

4=rigid gel, as 3 but more difficult to spread on skin

5=very rigid gel, difficult to spread and tends to fall off skin

The desirable level of clarity of the gel composition is between about 1 and about 2, more desirably between about 1 and about 1.5 based upon the scale detailed below. A clarity level of 1-1.5 represents a clear, transparent gel with negligible haziness. As mentioned above, the clearer the gel, the more stable its structure.

As it is difficult to measure clarity of a post-foaming gel when first dispensed because it will soon start to become cloudy upon release of the post-foaming agent and formation of the foam, the clarity of the gel is measured using a Gel Clarity Rating Scale.

The gel clarity test is conducted at an ambient temperature of about 20-25° C., where both the can and its contents are at this temperature. The can is held approximately 2 inches (5.1 cm) above a white sheet of feint ruled paper and actuated to dispense a 3 inch (7.6 cm) line of gel.

The characteristics of the gel are observed and scored for clarity using the following clarity rating scale:

C=1—Perfect clarity: clear gel, lines on paper easily defined

C=2—Standard clarity: clear gel, lines are visible

C=3—Minor clouding: hazy gel, lines not clearly defined

C=4—Major clouding: hazy gel, lines are not clearly visible

C=5—Critical clouding: cloudy gel, lines are obscured

It is desirable therefore that the formulation when dispensed forms a gel with a clarity rating score of C=1 or 2, more desirably C=1.

According to a further aspect of the invention, there is provided a method of manufacturing a post-foaming cleansing composition comprising an anionic surfactant, one or more non-ionic surfactants and a polymeric thickener, wherein the polymeric thickener is present in an amount of at least 0.3% by weight of the composition, wherein the anionic surfactant is present in the composition relative to the one or more non-ionic surfactants in a ratio of no more than 4.0:1, and wherein the one or more non-ionic surfactants includes a fatty acid alkanolamide, the method comprising the steps of:

i) providing an anionic surfactant, one or more non-ionic surfactants, a polymeric thickener and a post-foaming agent, wherein the one or more non-ionic surfactants includes a fatty acid alkanolamide; and

ii) mixing these components together.

According to one embodiment, the method of manufacturing a composition according to the invention may be in two stages. The first stage involves mixing together all ingredients, excluding the post-foaming agent, to produce a gel concentrate, and the second stage involves addition of the post-foaming agent into the gel concentrate to produce the complete composition.

The gel concentrate for the first stage may typically be manufactured as follows. According to one embodiment, the method of manufacturing a composition according to the invention may be as follows: The polymeric thickener is dispersed into water and fully dissolved. Optionally, the polymeric thickener may be pre-mixed with a non-aqueous liquid ingredient such as glycerine to produce a slurry which can be added into the water to facilitate rapid dispersion of the polymeric thickener thereby avoiding formation of gel lumps. The primary (anionic) surfactant is then added followed by the amphoteric surfactant, mixing until homogeneous. If a synthetic polymer is used, neutralisation to an appropriate pH will be required using a suitable base, such as sodium hydroxide or triethanolamine. The nonionic surfactants are then added. Finally, optional minor ingredients such as fragrance, skin conditioners, colouring agents, preservative and chelants are mixed in.

The second stage is the addition of the post-foaming agent into the gel concentrate. This requires very efficient mixing to ensure that the post-foaming agent is properly entrapped within the gel matrix to produce a uniform consistency. This can be achieved either in a batch process using a pressurised mixing vessel equipped with suitable means of agitation or alternatively as a continuous process whereby the post-foaming agent is introduced into the gel concentrate using an in-line dynamic or alternatively static mixer arrangement. The resulting gel is then pumped into suitable consumer packaging on a filling line.

According to an alternative aspect of the invention, the post-foaming agent may be added into the composition at an earlier stage of the manufacturing process.

According to a further aspect of the invention, there is provided a hair or skin cleansing product comprising a post-foaming cleansing composition as hereinabove described. There is also provided a shaving gel product comprising a post-foaming cleansing composition as hereinabove described.

According to a further aspect of the invention, there is provided a use of a post-foaming cleansing composition as hereinabove described in the cleansing of a person's skin or hair, or in the removal of a person's body hair by shaving.

According to a further aspect of the invention, there is provided a container comprising a post-foaming cleansing composition as hereinabove described. Suitable containers for housing the post-foaming cleansing composition may include an aerosol container, a bag on valve system, a bag in can system or an elasticated bladder container.

It will be appreciated that the post-foaming cleansing composition of the invention is not restricted to shower gels but is also applicable to other personal cleansing compositions such as hand wash, facial wash, shaving gels and shampoo compositions and the like.

The invention will now be described further by way of example with reference to the following examples which are intended to be illustrative only and in no way limiting upon the scope of the invention.

### EXAMPLE 1

In the compositions prepared according to the invention, the SLES (anionic surfactant), laureth-4 (non-ionic surfactant) and PEG-4 rapeseedamide (non-ionic surfactant) were set as three variables, while the rest of the components in the composition were kept constant. The components used and their amounts are shown below in Table 1.

TABLE 1

| Ingredient | | % w/w |
|---|---|---|
| Deionised Water | | Up to 100 |
| Disodium EDTA | Chelant | 0.10 |
| Citric Acid | pH adjuster | 0.10 |
| Capryl/Capramidopropyl Betaine 40% | Amphoteric | 2.00 |
| (Tegobetaine 810) | | |
| Lauramidopropyl Betaine (30%) | Amphoteric | 5.00 |
| Euxyl K400 | Preservative | 0.15 |
| Glycerin | Moisturiser | 0.10 |
| Ext D&C Violet No. 2 (0.1% Soln.) | Colourant | 0.80 |
| Dimethicone copolyol (ABIL B 88184) | Skin | 0.50 |
| | conditioning | |
| Fragrance | | 0.80 |
| PEG-7 Glyceryl Cocoate (Cetiol HE) | Non-ionic | 1.00 |
| Hydroxypropylmethyl Cellulose (Walocel | Thickener | 0.50 |
| HM4000) | | |
| Sodium Laureth Sulfate (SLES) | Anionic | Varies |
| Laureth-4 | Non-ionic | Varies |
| PEG-4 rapeseedamide (Amidet N) | Non-ionic | Varies |

Samples of gel concentrate were made up using the above ingredients and the resultant compositions are shown in Table 2 below. The figures refer to percentage inclusion in the gel concentrate by weight.

A post-foaming agent was then dosed into each of these examples using a laboratory mixing rig. In all cases the post-foaming agent was isopentane and the inclusion level was sufficient to provide a clear, rigid gel, typically within an 8-13% range based on the weight of the whole composition. The resulting gels were then transferred into consumer packs and assessed on dispensing for clarity and gel rigidity. The scores for these parameters are reported in Table 2 below.

TABLE 2

| | Anionic | Laureth- | Amidet | Cetiol | Total | Anionic: | | |
|---|---|---|---|---|---|---|---|---|
| Formula | (SLES) (%) | 4 (%) | N (%) | HE(%) | Nonionic | Nonionic | Clarity | Rigidity |
| A | 16.6 | 0.2 | 3.0 | 1.0 | 4.2 | 3.9:1 | 1 | 2.5 |
| B | 16.4 | 0.4 | 2.7 | 1.0 | 4.1 | 4:1 | 2 | 2.5 |
| C | 16.4 | 0.3 | 3.0 | 1.0 | 4.3 | 3.8:1 | 1.5 | 2.5 |
| D | 16.2 | 0.5 | 2.7 | 1.0 | 4.2 | 3.8:1 | 2 | 3 |
| E | 16.2 | 0.4 | 3.0 | 1.0 | 4.4 | 3.7:1 | 1 | 3 |
| F | 16.2 | 0.6 | 2.4 | 1.0 | 4.0 | 4:1 | 1 | 2.5 |
| G | 16.0 | 0.7 | 2.4 | 1.0 | 4.1 | 3.9:1 | 2 | 3 |
| H | 16.0 | 0.6 | 2.7 | 1.0 | 4.3 | 3.7:1 | 1 | 3 |
| I | 16.0 | 0.5 | 3.0 | 1.0 | 4.5 | 3.6:1 | 2 | 3 |
| J | 15.8 | 0.6 | 3.0 | 1.0 | 4.6 | 3.4:1 | 1 | 3 |
| K | 15.8 | 0.8 | 2.4 | 1.0 | 4.2 | 3.7:1 | 1 | 3 |
| L | 15.8 | 0.9 | 2.3 | 1.0 | 4.0 | 3.95:1 | 1 | 2 |
| M | 15.8 | 0.7 | 2.7 | 1.0 | 4.4 | 3.6:1 | 2 | 3 |
| N | 15.6 | 0.9 | 2.4 | 1.0 | 4.3 | 3.6:1 | 2 | 3 |
| O | 15.6 | 0.7 | 3.0 | 1.0 | 4.7 | 3.3:1 | 2 | 2.5 |
| P | 15.4 | 0.8 | 3.0 | 1.0 | 4.8 | 3.2:1 | 2 | 2.5 |
| Q | 15.4 | 1.0 | 2.4 | 1.0 | 4.4 | 3.5:1 | 2 | 2.5 |
| R | 15.0 | 1.0 | 3.0 | 1.0 | 5.0 | 3:1 | 2 | 2 |
| S | 15.0 | 1.2 | 2.4 | 1.0 | 4.4 | 3.3:1 | 2 | 2 |

Each of the above samples is a clear gel, stable, and possesses favourable rigidity properties which allow for a prolonged skin contact and therefore increased skin moisturisation and fragrance delivery characteristics.

It is of course to be understood that the present invention is not intended to be restricted to the foregoing examples which are described by way of example only.

1. A post-foaming cleansing composition comprising:
i) an anionic surfactant;
ii) one or more non-ionic surfactants;
iii) a polymeric thickener; and
iv) a post-foaming agent
wherein the polymeric thickener is present in an amount of 0.3% to 1.0% by weight of the composition; wherein the anionic surfactant is present in the composition relative to the one or more non-ionic surfactants in a ratio of 3.2:1 to 3.8:1; wherein the post-foaming agent is present in the composition in an amount of from 8 to 13% by weight of the composition; and wherein the one or more non-ionic surfactants includes a fatty acid alkanolamide.

2. A composition according to claim 1, wherein the fatty acid alkanolamide is a polyethylene glycol fatty acid amine.

3. A composition according to claim 2, wherein the polyethylene glycol fatty acid amine is polyethylene glycol-4 rapeseedamide.

4. A composition claim according to claim 1, wherein the polymeric thickener is present in an amount of 0.5% to 1% by weight of the composition.

5. A composition according to claim 1, wherein the anionic surfactant is present in the composition relative to the one or more non-ionic surfactants in a ratio of from about 3.3:1 to about 3.8:1.

6. A composition according to claim 1, wherein the polymeric thickener is one or more selected from the group consisting of a cellulose-based thickeners, natural gums and their chemically modified derivatives, and synthetic polymeric thickeners.

7. A composition according to claim 6, wherein the cellulose based thickeners comprise one or more selected from the group consisting of hydroxypropylmethyl cellulose, sodium carboxymethyl cellulose, hydroxyethyl cellulose, hydrophobically modified hydroxyethyl cellulose, hydroxypropyl cellulose; wherein the natural gums and their chemically modified derivatives comprise one or more selected from xanthan gum, carrageenan gum, gellan gum, hydroxypropyl guar and cationic guars; and wherein the synthetic polymeric thickeners comprise one or more selected from carbomers, acrylate copolymers, hydrophobically modified alkali soluble emulsion polymers, hydrophobically modified ethoxylated urethanes and polyethylene glycol (PEG)-based nonionic polymers.

8. A composition according to claim 1, wherein the anionic surfactant is one or more selected from the group consisting of alkali metal alkyl sulphates, alkali metal alkyl ether sulphates, sulfosuccinates, acyl glutamates, sultaines, taurates, carboxylates, isethionates, alkyl phosphates, sarcosinates, olefin sulphonates and alkyl polyglucoside esters.

9. A composition according to claim 1, wherein the non-ionic surfactant comprises one or more selected from the group consisting of laureth-4, PEG-4 rapeseedamide, alkoxylated alcohols, glyceryl esters, glycol esters, alkyl poly glucosides, alkoxylated carboxylic acids, alkanolamides, and derivatives thereof.

10. A composition according to claim 9, wherein the non-ionic surfactant comprises laureth-2, laureth-4, C12/13 pareth-3, ceteareth-4, or oleth-3, or glycol esters such as coconut fatty acid monoglyceride polyglycol ether or modified palm oil polyglycol ether.

11. A composition according to claim 1, comprising at least two non-ionic surfactants.

12. A composition according to claim 1, wherein the at least two non-ionic surfactants comprise laureth-4 and PEG-4 rapeseedamide.

13. (canceled)

14. A composition according to claim 1, wherein the post-foaming agent comprises one or more compounds selected from isopentane, n-pentane, isobutene or n-pentane.

15. A composition according to claim 1, further comprising an amphoteric surfactant.

16. A composition according to claim 15, wherein the amphoteric surfactant is one or more selected from the group consisting of capryl/capramidopropyl betaine, laurylamidopropyl betaine, cocoamidopropylbetaine, alkyl betaines, alkyl amine oxides, alkyl amidopropyl amine oxides, amphoacetates/diacetates, amphopropionates and alkyl amidopropyl hydroxysultaines.

17. A composition according to any preceding claim according to claim 1, further comprising one or more components selected from the group consisting of fragrances, skin conditioning agents, colouring agents, preservatives, antiseptic agents, chelating agents, sunscreen agents, plant extracts, pearlisers, opacifiers, shimmering agents, exfolliants, silicone oils, lipids, vitamins, skin lightening agents, or pH adjusters.

18. A composition according to claim 1 for use in cleansing of a person's skin or hair, or in shaving.

19. A product for cleansing a person's skin or hair, or for shaving, comprising a composition according to claim 1.

20. A method of manufacturing a composition according to claim 1, comprising the steps of:
i) providing an anionic surfactant, one or more non-ionic surfactants, a polymeric thickener and a post-foaming agent, wherein the one or more non-ionic surfactants includes a fatty acid alkanolamide; and
ii) mixing these components together.

21. A method of manufacturing a composition according to claim 20, comprising the steps of:
i) dispersing a polymeric thickener in water;
ii) adding an anionic surfactant to the dispersion and optionally an amphoteric surfactant;
iii) optionally adjusting the pH;
iv) adding one or more non-ionic surfactants; and
v) adding a post-foaming agent.

22. Use of a composition according to claim 1 in cleansing of a person's skin or hair, or in shaving.

23. A container comprising a composition according to claim 1.

24. A container according to claim 23, wherein the container is selected from an aerosol container, a bag on valve system, a bag in can system or an elasticated bladder container.

25. (canceled)
